SEQUENCE LISTING (8 sequences)

SEQ ID NO: 1 - protein, 587 aa, Artificial Sequence
  OhyLfus581Llac
MYYSNGNYEAFARPKKPEGVDGKSAYLIGSGLASLSAACFLIRDGQMKGENIHILEELDISGGSLDGILNPTRGFIIRGGREMEDHFECLWDLFRSIPSLEVDNASVLDEFYWLNKEDPNYSKCRLIKDRGQRLEDDGKFTLSDQSSEEMIKLFFTPEEKLEDKKITDVFSEEFFESNFWLYWSTMFAFEKWHSAIEMRRYIMRFIHHIEGLPDFSALKFTKYNQYESLVKPLISYLKDHGVDFKYGAQVENVEVDFSNGKKVAKAIVFADKKIKELTGNDLVFVTNGSITESSTQGSPTQVAPKTSELGGSWKLWQNLAKQSEEFGHPDVFCKDIPKEAWVVSATVTWKNLKIQPYFEKLTHRQLRSGKVVTGGIVTVKDSNWLLSFTTHRQPHFKEQNDQETVTWVYGLLSNTPGNYIKKPIEDCTGEEITQELLYHLGMPEEEIEAFVKENTNTIPVYMPFITSYFMLREPGDRPLVVPEQSVNLAFLGNFAETERDTVFTTEYSVRTAMEAVYQLLNVDRGVPEVYASAYDLRTLASSAYYLSDKQKLEDLELSFIKKQALKVGLKKIKGTYIEELLEDAGLI

SEQ ID NO: 2 - DNA, 1764 bp, Artificial Sequence
  OhyLfus581Llac
atgtattacagcaatggtaactatgaagctttcgcacgtcctaaaaaaccggagggagtagatggaaaatctgcttatctcattggttcgggcctagcctcgctgtcagcagcatgttttttaattcgtgatggccaaatgaaaggtgagaacattcatattctggaagagctagatatttcaggaggcagtcttgatggcatacttaatccgacgagaggatttattattcgcggtggtcgagaaatggaagatcattttgaatgcttatgggatttattccgttccattccttccttagaggtagacaatgcttctgtgttagatgaattttattggttaaacaaagaggaccccaattattcaaaatgtcgtttaataaaagatagaggtcaaagacttgaagatgatggcaagtttactttatcggatcagtcatctgaggaaatgattaaattattctttacacctgaggaaaagttagaggataaaaaaattacagatgttttctcagaggaattttttgaatcgaacttttggctgtattggtccactatgtttgcgtttgaaaaatggcattcagctattgagatgcgtcgttacatcatgcgtttcatccaccatattgaggggttacctgatttttcggccctaaaatttacaaaatataatcaatatgaatcgctagtaaaaccattgatttcatacttaaaagatcatggagttgattttaaatatggagcacaggttgaaaatgttgaagttgacttttcaaatggtaaaaaggtagcaaaagctattgtatttgctgacaaaaaaataaaagaactgacaggaaatgacttagtttttgtcacaaatggttcaattacagaaagttcaactcaaggttcaccaacacaagtcgctccgaaaaccagtgaacttggtggctcttggaaactttggcaaaatttagctaaacaatcagaagaatttggacatccagatgttttttgtaaggacattccaaaagaagcttgggttgtatcagcaactgtcacttggaaaaatttaaaaattcaaccttattttgaaaaattaacacatcgtcaattgcgttcaggaaaagttgtcacaggaggaattgttactgttaaagattcaaattggctcctttcatttacaactcaccgccaacctcattttaaagaacaaaatgaccaagaaacagtaacttgggtttatggtcttctttcaaatacacctgggaactatatcaaaaaaccaatcgaagattgtacaggtgaagaaattactcaagaattactttatcatttaggaatgccagaagaagaaattgaagcatttgttaaagaaaatactaatacaatcccagtatatatgcctttcatcacttcttatttcatgcttcgtgaaccaggggatcggcctcttgtagtacctgaacaatcagtcaatttagcgtttttaggaaattttgcggaaactgagcgtgatacagtctttacaaccgagtattcagtcagaactgcgatggaagctgtttatcaattattaaatgttgatcgcggtgttccggaagtctatgcttcggcttatgatttacgaactttagcaagttcagcctactacctatcagataagcaaaaattagaagatttagagctctcatttattaaaaaacaagcgctcaaagtgggacttaagaaaatcaaaggaacatatattgaagaacttctagaagatgcgggtctgatttaa

SEQ ID NO: 3 - protein, 587 aa, Lactococcus lactis
  Oleat Hydratase
MRYTNGNYEAFVHPRKPENVDQKSAYIIGAGLAGLATAVFLIRDGQMSGDRIHILEELPLSGGSLDGSFIPHDGFVVRGGREMENHFECLWDLFRTIPSLEVDNASVLDEFYRIDRDDPNSSNCRIIHNRGERVDDDGKFTLSPKAQEEIIKLFMTKEELLVGKTIEDCFTDEFFESNFWLYWCSMFAFEKWHSAIEMRRYIMRFIHHIEGLPDFSALKFTKYNQYESLVKPLISYLKDHGVDFKYGAQVENVEVDFSNGKKVAKAIVFADKKIKELTGNDLVFVTNGSITESSTQGSPTQVAPKTSELGGSWKLWQNLAKQSEEFGHPDVFCKDIPKEAWVVSATVTWKNLKIQPYFEKLTHRQLRSGKVVTGGIVTVKDSNWLLSFTTHRQPHFKEQNDQETVTWVYGLLSNTPGNYIKKPIEDCTGEEITQELLYHLGMPEEEIEAFVKENTNTIPVYMPFITSYFMLREPGDRPLVVPEQSVNLAFLGNFAETERDTVFTTEYSVRTAMEAVYQLLNVDRGVPEVYASAYDLRTLASSAYYLSDKQKLEDLELSFIKKQALKVGLKKIKGTYIEELLEDAGLI

SEQ ID NO: 4 - DNA, 1764 bp, Lactococcus lactis
  Oleat Hydratase
atgcgttatacaaatggaaattatgaagctttcgttcacccaagaaaaccagaaaatgttgatcaaaaatcagcttatattattggggcaggccttgcagggcttgcaacagcagtatttttaattcgtgacggtcaaatgtcaggggaccgtattcatattttagaagagttaccgttgtcaggaggttctcttgacggttcatttattccacatgatggtttcgttgttcgtggtggacgtgaaatggaaaaccattttgaatgtctttgggacttattccgaacaattccgtcattagaagtggacaatgcgtctgttttagatgaattttatcgaattgaccgggatgacccaaattcatctaactgtcgtattatccataatcgtggtgaacgggttgatgatgatggaaaatttaccctaagtccaaaagctcaagaagaaataatcaaattattcatgactaaagaagaactacttgtaggtaaaacaatagaagattgctttactgatgagttcttcgaatcaaatttttggctttattggtgttcaatgtttgcttttgaaaaatggcattcagctattgagatgcgtcgttacatcatgcgtttcatccaccatattgaggggttacctgatttttcggccctaaaatttacaaaatataatcaatatgaatcgctagtaaaaccattgatttcatacttaaaagatcatggagttgattttaaatatggagcacaggttgaaaatgttgaagttgacttttcaaatggtaaaaaggtagcaaaagctattgtatttgctgacaaaaaaataaaagaactgacaggaaatgacttagtttttgtcacaaatggttcaattacagaaagttcaactcaaggttcaccaacacaagtcgctccgaaaaccagtgaacttggtggctcttggaaactttggcaaaatttagctaaacaatcagaagaatttggacatccagatgttttttgtaaggacattccaaaagaagcttgggttgtatcagcaactgtcacttggaaaaatttaaaaattcaaccttattttgaaaaattaacacatcgtcaattgcgttcaggaaaagttgtcacaggaggaattgttactgttaaagattcaaattggctcctttcatttacaactcaccgccaacctcattttaaagaacaaaatgaccaagaaacagtaacttgggtttatggtcttctttcaaatacacctgggaactatatcaaaaaaccaatcgaagattgtacaggtgaagaaattactcaagaattactttatcatttaggaatgccagaagaagaaattgaagcatttgttaaagaaaatactaatacaatcccagtatatatgcctttcatcacttcttatttcatgcttcgtgaaccaggggatcggcctcttgtagtacctgaacaatcagtcaatttagcgtttttaggaaattttgcggaaactgagcgtgatacagtctttacaaccgagtattcagtcagaactgcgatggaagctgtttatcaattattaaatgttgatcgcggtgttccggaagtctatgcttcggcttatgatttacgaactttagcaagttcagcctactacctatcagataagcaaaaattagaagatttagagctctcatttattaaaaaacaagcgctcaaagtgggacttaagaaaatcaaaggaacatatattgaagaacttctagaagatgcgggtctgatttaa

SEQ ID NO: 5 - protein, 590 aa, Lysinibacillus fusiformis
  Oleat Hydratase
MYYSNGNYEAFARPKKPEGVDGKSAYLIGSGLASLSAACFLIRDGQMKGENIHILEELDISGGSLDGILNPTRGFIIRGGREMEDHFECLWDLFRSIPSLEVDNASVLDEFYWLNKEDPNYSKCRLIKDRGQRLEDDGKFTLSDQSSEEMIKLFFTPEEKLEDKKITDVFSEEFFESNFWLYWSTMFAFEKWHSAMEMRRYIMRFIHHIGGLPDLSALKFTKYNQYESLVLPMIKYLEGHDVDFQYNTVVENVLVDKVGDKKVAHTLVLRKNGVKENIELTENELVFVTNGSITESTTYGDNYTPAPVNKELGGSWSLWKNIAAQDTDFGRPEKFCDNLPKESWFVSATLTTLDDRVAPYIEKISKRDPYAGKVVTGGIVTATDSNWMLSYTLNRQPHFKHQPKDQLVVWIYGLLSNKPGDFIKKSITECSGIEIAQEWLYHMGVPVDEIPDIAQNSCNTIPCYMPYITSYFMPRAMGDRPLVVPEGSANLAFIGNFSETVRDTVFTTEYSVRTAMEAVYQLLNIDRGVPEVFASSFDVRTLLASTARLLDGKKLTDIDAPFILKQIGKLGIHKTKDTIIYDLLKESELI

SEQ ID NO: 6 - DNA, 1773 bp, Lysinibacillus fusiformis
  Oleat Hydratase
atgtattacagcaatggtaactatgaagctttcgcacgtcctaaaaaaccggagggagtagatggaaaatctgcttatctcattggttcgggcctagcctcgctgtcagcagcatgttttttaattcgtgatggccaaatgaaaggtgagaacattcatattctggaagagctagatatttcaggaggcagtcttgatggcatacttaatccgacgagaggatttattattcgcggtggtcgagaaatggaagatcattttgaatgcttatgggatttattccgttccattccttccttagaggtagacaatgcttctgtgttagatgaattttattggttaaacaaagaggaccccaattattcaaaatgtcgtttaataaaagatagaggtcaaagacttgaagatgatggcaagtttactttatcggatcagtcatctgaggaaatgattaaattattctttacacctgaggaaaagttagaggataaaaaaattacagatgttttctcagaggaattttttgaatcgaacttttggctgtattggtccactatgtttgcgtttgaaaaatggcattctgcaatggaaatgcgccgttatattatgcgtttcattcaccatattggtggattaccagatctatcagcattgaaatttacaaaatataaccaatatgaatcgttagtgctaccgatgataaaatatttagaaggtcatgatgttgattttcaatataatacggtagtagagaatgttttggtcgataaagtgggcgacaaaaaagtagctcatacattagttttaagaaaaaatggtgtgaaagagaatattgagctaacagaaaatgaattggtatttgtgacaaatggtagtattacggagagtacaacatacggggataattataccccagcccctgtcaataaagaattaggtggaagctggtcactgtggaaaaacatcgctgcccaagatactgactttggacgaccagaaaagttctgtgataatcttccaaaagaaagctggtttgtttccgccactctaacgactttagacgatcgtgtggctccttatattgaaaaaattagtaaaagggacccgtatgcaggcaaagtagtaacaggaggtattgtcacagctacagattctaattggatgctaagctatacgttaaaccgacaacctcattttaaacatcaaccaaaagaccaattagtcgtttggatttatggtttactatcaaataaaccaggtgattttattaaaaaaagcattaccgaatgttcaggtattgagatagcacaagagtggttgtaccatatgggtgtaccggttgatgaaattccagacatcgcacaaaattcttgtaacacgattccatgctatatgccttacatcacctcttatttcatgccaagagccatgggggatcgtccattagttgtgccagaagggtctgctaatttagccttcattggcaactttagtgaaactgtaagagataccgtttttactactgaatattcagtaagaactgcgatggaggcagtctatcaattattaaatattgatcgtggagttccagaagtattcgcatcttcttttgatgttcgcacattattagcttcaactgctcgcttattagatggcaaaaaattaacagatattgatgcgccatttattttaaaacaaattggtaaattaggaattcacaaaacaaaagatacaattatttatgacttactgaaagaaagtgaattaatatag

SEQ ID NO: 7 - protein, 590 aa, Artificial Sequence
  OhyLlac579Lfus
MRYTNGNYEAFVHPRKPENVDQKSAYIIGAGLAGLATAVFLIRDGQMSGDRIHILEELPLSGGSLDGSFIPHDGFVVRGGREMENHFECLWDLFRTIPSLEVDNASVLDEFYRIDRDDPNSSNCRIIHNRGERVDDDGKFTLSPKAQEEIIKLFMTKEELLVGKTIEDCFTDEFFESNFWLYWCSMFAFEKWHSAMEMRRYIMRFIHHIGGLPDLSALKFTKYNQYESLVLPMIKYLEGHDVDFQYNTVVENVLVDKVGDKKVAHTLVLRKNGVKENIELTENELVFVTNGSITESTTYGDNYTPAPVNKELGGSWSLWKNIAAQDTDFGRPEKFCDNLPKESWFVSATLTTLDDRVAPYIEKISKRDPYAGKVVTGGIVTATDSNWMLSYTLNRQPHFKHQPKDQLVVWIYGLLSNKPGDFIKKSITECSGIEIAQEWLYHMGVPVDEIPDIAQNSCNTIPCYMPYITSYFMPRAMGDRPLVVPEGSANLAFIGNFSETVRDTVFTTEYSVRTAMEAVYQLLNIDRGVPEVFASSFDVRTLLASTARLLDGKKLTDIDAPFILKQIGKLGIHKTKDTIIYDLLKESELI

SEQ ID NO: 8 - DNA, 1773 bp, Artificial Sequence
  OhyLlac579Lfus
atgcgttatacaaatggaaattatgaagctttcgttcacccaagaaaaccagaaaatgttgatcaaaaatcagcttatattattggggcaggccttgcagggcttgcaacagcagtatttttaattcgtgacggtcaaatgtcaggggaccgtattcatattttagaagagttaccgttgtcaggaggttctcttgacggttcatttattccacatgatggtttcgttgttcgtggtggacgtgaaatggaaaaccattttgaatgtctttgggacttattccgaacaattccgtcattagaagtggacaatgcgtctgttttagatgaattttatcgaattgaccgggatgacccaaattcatctaactgtcgtattatccataatcgtggtgaacgggttgatgatgatggaaaatttaccctaagtccaaaagctcaagaagaaataatcaaattattcatgactaaagaagaactacttgtaggtaaaacaatagaagattgctttactgatgagttcttcgaatcaaatttttggctttattggtgttcaatgtttgcttttgaaaaatggcattctgcaatggaaatgcgccgttatattatgcgtttcattcaccatattggtggattaccagatctatcagcattgaaatttacaaaatataaccaatatgaatcgttagtgctaccgatgataaaatatttagaaggtcatgatgttgattttcaatataatacggtagtagagaatgttttggtcgataaagtgggcgacaaaaaagtagctcatacattagttttaagaaaaaatggtgtgaaagagaatattgagctaacagaaaatgaattggtatttgtgacaaatggtagtattacggagagtacaacatacggggataattataccccagcccctgtcaataaagaattaggtggaagctggtcactgtggaaaaacatcgctgcccaagatactgactttggacgaccagaaaagttctgtgataatcttccaaaagaaagctggtttgtttccgccactctaacgactttagacgatcgtgtggctccttatattgaaaaaattagtaaaagggacccgtatgcaggcaaagtagtaacaggaggtattgtcacagctacagattctaattggatgctaagctatacgttaaaccgacaacctcattttaaacatcaaccaaaagaccaattagtcgtttggatttatggtttactatcaaataaaccaggtgattttattaaaaaaagcattaccgaatgttcaggtattgagatagcacaagagtggttgtaccatatgggtgtaccggttgatgaaattccagacatcgcacaaaattcttgtaacacgattccatgctatatgccttacatcacctcttatttcatgccaagagccatgggggatcgtccattagttgtgccagaagggtctgctaatttagccttcattggcaactttagtgaaactgtaagagataccgtttttactactgaatattcagtaagaactgcgatggaggcagtctatcaattattaaatattgatcgtggagttccagaagtattcgcatcttcttttgatgttcgcacattattagcttcaactgctcgcttattagatggcaaaaaattaacagatattgatgcgccatttattttaaaacaaattggtaaattaggaattcacaaaacaaaagatacaattatttatgacttactgaaagaaagtgaattaatatag

(19) United States
(12) Patent Application Publication
(10) Pub. No.: US 2022/0282288 A1
(43) Pub. Date: Sep. 8, 2022

(54) NOVEL OLEATE HYDRATASES

(71) Applicant: BRAIN BIOTECH AG, Zwingenberg (DE)

(72) Inventors: Birgit BORGARDS, Mannheim (DE); Patrick LORENZ, Lorsch (DE)

(21) Appl. No.: 17/634,342

(22) PCT Filed: Aug. 14, 2020
(86) PCT No.: PCT/EP2020/072838

§ 371 Date: Feb. 10, 2022

(30) Foreign Application Priority Data
Aug. 14, 2019 (EP) 19191723.6

(51) Int. Cl. C12P 7/6409; C12N 15/52; C12N 15/62; C12N 9/14; C12N 15/63; C12N 9/10

(52) U.S. Cl. CPC C12P 7/6409; C12N 15/52; C12N 15/62; C12Y 402/01053; C12N 9/14; C12N 15/63; C12N 9/10

(57) ABSTRACT

The present invention relates to a method of producing a 10-hydroxy fatty acid, wherein the method comprises contacting a sample comprising a (9Z) or (9E)-fatty acid with a polypeptide having the activity of an oleate hydratase (EC 4.2.1.53) encoded by a nucleic acid molecule, wherein the nucleic acid molecule is (a) a nucleic acid molecule encoding a polypeptide comprising or consisting of the amino acid sequence of SEQ ID NO: 1 or 7; (b) a nucleic acid molecule comprising or consisting of the nucleotide sequence of SEQ ID NO: 2 or 8; (c) a nucleic acid molecule comprising or consisting of a nucleic acid molecule encoding a polypeptide having the activity of an oleate hydratase the amino acid sequence of which is at least 91% identical to the amino acid sequence of SEQ ID NO: 1 or 7; (d) a nucleic acid molecule encoding a polypeptide having the activity of an oleate hydratase and comprising or consisting of a nucleotide sequence which is at least 91% identical to the nucleotide sequence of SEQ ID NO: 2 or 8; (e) a fragment of the nucleic acid molecule of any of (a) to (d) comprising at least 1341 nucleotides and encoding a polypeptide having the activity of an oleate hydratase; or (f) the nucleic acid sequence of any of (a) to (d) wherein T is U.

### RELATED PATENT APPLICATIONS

This patent application is a 35 U.S.C. 371 national phase patent application of PCT/EP2020/072838 filed on Aug. 14, 2020, entitled “NOVEL OLEATE HYDRATASES”, naming Birgit Borgards and Patrick Lorenz as inventors, and designated by attorney docket no. AC1261 PCT, which claims priority to European Application No. 19191723.6 filed on Aug. 14, 2019, entitled “NOVEL OLEATE HYDRATASES,” naming Birgit Borgards and Patrick Lorenz as inventors, and designated by attorney docket no. AC1261 EP. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy is named Sequence_Listing and is 32 kilobytes in size.

The present invention relates to a method of producing a 10-hydroxy fatty acid, wherein the method comprises contacting a sample comprising a (9Z) or (9E)-fatty acid with a polypeptide having the activity of an oleate hydratase (EC 4.2.1.53) encoded by a nucleic acid molecule, wherein the nucleic acid molecule is (a) a nucleic acid molecule encoding a polypeptide comprising or consisting of the amino acid sequence of SEQ ID NO: 1 or 7; (b) a nucleic acid molecule comprising or consisting of the nucleotide sequence of SEQ ID NO: 2 or 8; (c) a nucleic acid molecule comprising or consisting of a nucleic acid molecule encoding a polypeptide having the activity of an oleate hydratase the amino acid sequence of which is at least 91% identical to the amino acid sequence of SEQ ID NO: 1 or 7; (d) a nucleic acid molecule encoding a polypeptide having the activity of an oleate hydratase and comprising or consisting of a nucleotide sequence which is at least 91% identical to the nucleotide sequence of SEQ ID NO: 2 or 8; (e) a fragment of the nucleic acid molecule of any of (a) to (d) comprising at least 1341 nucleotides and encoding a polypeptide having the activity of an oleate hydratase; or (f) the nucleic acid sequence of any of (a) to (d) wherein T is U.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Fatty acids differ from each other in the length and in the number and position of the double bonds. Natural fatty acids usually consist of an even number of carbon atoms and are unbranched. Fatty acids with one or more double bonds are called unsaturated fatty acids, with the double bond usually being in the [omega]-configuration.

Fats and oils have gained considerable interest in the last years, because they are some of the most important renewable raw materials for the chemical industry. To adapt the fatty acids to the different uses, modifications have been introduced to the fatty acids. While most of the modifications were earlier directed to the carboxyl group of the fatty acid, in recent years also the alkyl chain of the fatty acids has been modified to obtain important starting materials for the fine chemical industry.

One of these modification reactions is the addition of water to the double bonds of unsaturated fatty acids, for example, by fatty acid hydratases leading to the formation of hydroxy fatty acids. Hydroxy fatty acids can be used as lubricants, surfactants and plasticizers; as components in detergent, coating and paint industries; and in the synthesis of resins (WO 2008/119735). Furthermore, short-chain hydroxy fatty acids and lactones made from these fatty acids can be used as flavor ingredients and/or fragrance ingredients. The lactones possess various sensory properties with mainly fruity and fatty characteristics, which make them interesting food additives. One of the main products derived from aroma biotechnology is [gamma]-decalactone which can be obtained by biotransformation of the long-chain hydroxy fatty acid precursor by yeast cells.

For the hydration reaction of unsaturated fatty acids both a chemical reaction and microbes can be used. The chemical addition of water has the disadvantage that it is neither regioselective nor stereoselective. The lack of regioselectivity means that all carbon atoms of the double bonds are hydrated with essentially the same efficiency, leading to the formation of a mixture of hydrated products which have to be separated from each other after completion of the reaction. The term “stereoselectivity” means that of a compound present in both a cis- and a trans-configuration, only one of these configurations of the compound is modified. In contrast, microbial hydration is both regioselective and stereoselective. Microbial water attachment to unsaturated fatty acids was observed with Pseudomonas species, as well as with the bacterial genera Nocardia, Rhodococcus, Corynebacterium and Micrococcus (Wallen et al. (1962) Arch. Biochem. Biophys. 99: 249-253; Koritala et al. (1989) Appl. Microbiol. Biotechnol. 32: 299-304; Seo et al. (1981) Agric. Biol. Chem. 45: 2025-2030; Blank et al. (1991) Agric. Biol. Chem. 55: 2651-2652). Furthermore, hydroxy fatty acids could also be obtained with the yeast Saccharomyces cerevisiae (EI-Sharkawy et al. (1992) Appl. Environ. Microbiol. 58: 2116-2122). However, use of this microorganism does not involve the use of a purified enzyme, but rather the use of a cell extract.

Fatty acid hydratases have been described from different organisms, e.g. from Streptococcus pyogenes (WO 2008/119735) and from Elizabethkingia meningoseptica (Bevers et al., loc. cit.; GenBank Accession number GQ144652). Originally, most of these proteins were annotated as myosin-cross-reactive antigen due to their homology to the Streptococcus pyogenes 67.5 kDa protein which had later been found to have fatty acid hydratase activity (WO 2008/119735).

Fatty acid hydratase is an enzyme that catalyzes the conversion of oleic acid (OA) into 10-hydroxystearic acid (10-HSA) and is therefore also referred to as an oleate hydratase. The enzymatic hydration of carbon-carbon double bonds was first reported by Bevers et al. in 2009 (Bevers, Loes E. et al. (2009) J. Bacteriol., 191, 5010-5012). This enzyme was isolated from Elizabethkinga meningoseptica (formerly known as Pseudomonas sp. 3266), subsequently cloned and expressed in E. coli. Since the first fatty acid hydratase was described, a variety of microorganisms including other Pseudomonas sp. strains and species of Nocardia (Rhodococcus), Corynebacterium, Spingobacterium, Micrococcus, Macrococcus, Aspergillus, Candida, Mycobacterium and Schizosaccharomyces followed (WO 2016/151115).

A recent report showed that the fatty acid hydratase from Lysinibacillus fusiformis catalyzes the hydration of oleic acid with the hitherto highest reported activity. The fatty acid hydratase from Lysinibacillus fusiformis also showed activity against a number of other unsaturated fatty acids with a length of C14 to C18 with a cis C9-C10 double bond, for example, myristoleic acid (C14), palmitoleic acid (C16), linoleic acid (C18), a-linolenic acid (C18) and y-linolenic acid (KR 101749429 B1; Kim, Bi-Na et al. (2012) Appl. Microbiol. Biotechnol. 95, 929-937).

Conversion of oleic acid to 10-HSA using whole cells of recombinant E. coli containing fatty acid hydratase from Stenotrophomonas maltophilia has been reported by Joo et al. (Joo, Young-Chul et al. (2012) J.Biotechnol. 158, 17-23). The same group also expressed a putative fatty acid hydratase from Macrococcus caseolyticus in Escherichia coli. The FAD dependent enzyme catalyzes hydration at the cis-9-double and cis-12-double bonds of unsaturated fatty acids (Joo, Young-Chul et al. (2012) Biochimie 94, 907-915). Maximum enzyme activity with oleic acid as substrate was reported at pH 6.5 and 25° C. with 2% (v/v) ethanol and 0.2 mM FAD.

Heo et al. used Flavobacterium sp. strain DS5 (NRRL B-14859) to convert two vegetable oils, olive oil and soybean oil, directly to oxygenated fatty acids such as 10-ketostearic acid and 10-hydroxystearic acid (Heo, Shin-Haeng et al. (2009) N. Biotechnol. 26, 105-108).

Kang et al. categorized fatty acid hydratases as either OhyA1 or OhyA2 based on the activities of the holoenzymes upon adding cofactors, which were determined by the type of the fourth conserved amino acid of the flavin adenine dinucleotide (FAD)-binding motif. The activity of OhyA1 showed an increase by adding cofactors, whereas the activity of the OhyA2 as a holoenzyme was not affected by the addition of cofactors (Kang, Woo-Ri et al. (2017) Appl. Environ. Microbiol. 83, e03351-16).

Hence, although several fatty acid hydratases are known from the art there is still an ongoing need for new fatty acid hydratases that can be used for the production of modified fatty acids in view of their importance in chemical industry and for the production of various goods. This need is adressed by the present invention.

Accordingly, the present invention relates in a first aspect to a method of producing a 10-hydroxy fatty acid, wherein the method comprises contacting a sample comprising a (9Z) or (9E)-fatty acid with a polypeptide having the activity of an oleate hydratase (EC 4.2.1.53) encoded by a nucleic acid molecule, wherein the nucleic acid molecule is (a) a nucleic acid molecule encoding a polypeptide comprising or consisting of the amino acid sequence of SEQ ID NO: 1 or 7; (b) a nucleic acid molecule comprising or consisting of the nucleotide sequence of SEQ ID NO: 2 or 8; (c) a nucleic acid molecule comprising or consisting of a nucleic acid molecule encoding a polypeptide having the activity of an oleate hydratase the amino acid sequence of which is at least 80%, preferably at least 91% identical to the amino acid sequence of SEQ ID NO: 1 or 7; (d) a nucleic acid molecule encoding a polypeptide having the activity of an oleate hydratase and comprising or consisting of a nucleotide sequence which is at least 80%, preferably at least 91% identical to the nucleotide sequence of SEQ ID NO: 2 or 8; (e) a fragment of the nucleic acid molecule of any of (a) to (d) comprising at least 1341 nucleotides and encoding a polypeptide having the activity of an oleate hydratase; or (f) the nucleic acid sequence of any of (a) to (d) wherein T is U.

Among the amino acid sequences of SEQ ID NOs 1 and 7 SEQ ID NO: 1 is preferred, and among the nucleotide sequences of SEQ ID NOs 2 and 8 SEQ ID NO: 2 is preferred.

A fatty acid is a carboxylic acid with a long aliphatic chain, which is either saturated or unsaturated. Saturated fatty acids have no C═C double bonds. They have the same formula CH₃(CH₂)ₙCOOH, with variations in “n”. An important example of a saturated fatty acid is stearic acid (n=16), which when neutralized with lye is the most common form of soap. The numbering of the carbon atoms within the fatty acid molecules starts from the carbon atom of the carboxyl group of the fatty acid.

On the other hand, unsaturated fatty acids have one or more C═C double bonds. The C═C double bonds can give either cis (or Z) or trans (or E) isomers. A cis (or Z) configuration means that the two hydrogen atoms adjacent to the double bond stick out on the same side of the chain. The rigidity of the double bond freezes its conformation and, in the case of the cis isomer, causes the chain to bend and restricts the conformational freedom of the fatty acid. A trans (or E) configuration, by contrast, means that the adjacent two hydrogen atoms lie on opposite sides of the chain. As a result, they do not cause the chain to bend much, and their shape is similar to straight saturated fatty acids. An example of an unsaturated fatty acid with a cis configuration is oleic acid having the IUPAC name (9Z)-octadecenoic acid. Oleic acid has a cis double bond between the carbon atoms 9 and 10. An example of an unsaturated fatty acid with a trans configuration is elaidic acid having the IUPAC name (9E)-octadecenoic acid. Elaidic acid has a trans double bond between the carbon atoms 9 and 10. Further examples for unsaturated fatty acids include lauroleic acid (C 12:1), myristoleic acid (C14:1), palmitoleic acid (C16:1), linoleic acid (C18:2), alpha- linolenic acid (C18:3), arachidonic acid (20:4), eicosapentaenoic acid (C20:5), erucic acid (C22:1) and docosahexaenoic acid (C22:6).

Naturally occurring fatty acids generally have an unbranched chain of an even number of carbon atoms, from 4 to 28. Short-chain fatty acids (SCFA) are fatty acids with aliphatic tails of five or fewer carbons (e.g. butyric acid). Medium-chain fatty acids (MCFA) are fatty acids with aliphatic tails of 6 to 12 carbons. Long-chain fatty acids (LCFA) are fatty acids with aliphatic tails of 13 to 21 carbons. Very long chain fatty acids (VLCFA) are fatty acids with aliphatic tails of 22 or more carbons.

Hydroxy fatty acids are formed from unsaturated fatty acids by hydration, i.e. water addition to the double bond, which means that one carbon atom of the double bond contains a hydroxy group and one carbon atom of the double bond contains a hydrogen atom after the hydration reaction.

A 10-hydroxy fatty acid is a fatty acid having a single bond between its carbon atoms 9 and 10 and a hydroxyl group at carbon atom 10. A (9Z)-fatty acid is a fatty acid having a cis double bond between its carbon atoms 9 and 10. A (9E)-fatty acid is a fatty acid having a trans double bond between its carbon atoms 9 and 10.

In accordance with the present invention the term “nucleic acid molecule” defines a linear molecular chain. The specific nucleic acid molecule in accordance with the invention consists of at least 1341 nucleotides. The group of molecules designated herein as “nucleic acid molecules” also comprises complete genes. The term “nucleic acid molecule” is interchangeably used herein with the term “polynucleotide”.

Nucleic acid molecules in accordance with the present invention include DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases. Included are also single- and double-stranded hybrid molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphorarnidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may additionally comprise promoters, enhancers, response elements, signal sequences, polyadenylation sequences, introns, 5′- and 3′- non-coding regions, and the like.

The term “polypeptide” as used herein interchangeably with the term “protein” describes linear molecular chains of amino acids, including single chain proteins or their fragments. Polypeptides may further form oligomers consisting of at least two identical or different molecules. The corresponding higher order structures of such multimers are, correspondingly, termed homo- or heterodimers, homo- or heterotrimers etc. The polypeptides of the invention may form heteromultimers or homomultimers, such as heterodimers or homodimers. Furthermore, peptidomimetics of such proteins/polypeptides where amino acid(s) and/or peptide bond(s) have been replaced by functional analogues are also encompassed by the invention. Such functional analogues include all known amino acids other than the 20 gene-encoded amino acids, such as selenocysteine. The terms “polypeptide” and “protein” also refer to naturally modified polypeptides and proteins where the modification is effected e.g. by glycosylation, acetylation, phosphorylation, ubiquitinylation and similar modifications which are well known in the art.

An oleate hydratase (EC 4.2.1.53) (or fatty acid hydratase) also designated a “polypeptide having the activity of an oleate hydratase (EC 4.2.1.53)” disclosed herein is an enzyme which is capable of catalyzing the conversion of a (9Z) or (9E)-fatty acid into a 10-hydroxy fatty acid by the nucleophilic addition of a water to the (9Z) or (9E) double bond. For instance, the reaction oleic acid+H₂O→(R)-10-hydroxystearic acid (FIG. 1.) is catalyzed by said enzyme. The substrate is preferably a (9Z)-fatty acid.

Whether a given polypeptide has the activity of an oleate hydratase (EC 4.2.1.53) or not can be tested by well established methods. The fatty acid hydratase activity can be measured by incubating the polypeptide with the corresponding substrate, in particular an unsaturated fatty acid substrate as described herein, under appropriate conditions and analyzing the reaction products, e.g. by GC-MS analysis. In case reaction products comprise 10-HSA, the polypeptide has the activity of an oleate hydratase (EC 4.2.1.53) and in case the reaction products do not comprise 10-HSA, the polypeptide does not have the activity of an oleate hydratase (EC 4.2.1.53).

For example, the enzymatic activity of converting oleic acid (OA) into 10-hydroxystearic acid (10-HSA) via hydration can be measured by incubating the enzyme with oleic acid and analyzing the reaction products, e.g. via gas chromatography. It can in particular be assayed by an assay as described in Bevers et al. (J. Bacterid. 191 (2009), 5010-5012). In brief, the enzyme is incubated for at least 1 h in 20 mM Tris (pH 8)-50 mM NaCl with oleic acid at an appropriate temperature at which the enzyme shows activity (e.g., 22° C., 30° C. or 37° C.). The reaction is stopped by the addition of 3M HCl and the products are analyzed by gas chromatography (GC). The occurrence of 10-HSA is indicative of fatty acid hydratase activity. Alternatively, the activity can also be tested as described in WO 2008/119735. In this assay 20 μg oleic acid is mixed with the enzyme to be tested in 1 ml 0.1 M sodium phosphate pH 7.1; 0.1 M NaCl and incubated for at least 1 h at an appropriate temperature (e.g., 22° C., 30° C. or 37° C.). Subsequently the products are characterized by GC (see Example 6 of WO 2008/119735). The skilled person can devise additional methods without further ado.

The oleate hydratase of the present invention has regioselectivity, as it only hydrates the carbon atom at position 10 of the fatty acids and not the carbon atom at position 9 of the fatty acids. This leads to the production of 10-hydroxy fatty acids. Moreover, only substrates possessing a double bond in the c[omega]-configuration are hydrated by the enzyme. Hence, the oleate hydratase of the present invention specifically produces 10-hydroxy fatty acids in which the hydroxy group is located on carbon atom 10 of the fatty acid. For example, 10-hydroxyhexadecanoic acid is produced from palmitoleic acid, 10-hydroxyoctadecanoic acid is produced from oleic acid, 10-hydroxy-(9Z)-octadec-9-enoic acid is produced from linoleic acid and 10-hydroxy-12Z,15Z-octadeca-12,15-dienoic acid is produced from [alpha]-linolenic acid.

The (9Z) or (9E) unsaturated fatty acids which are to be reacted to the hydroxy fatty acids may be used in pure form or in the form of their natural precursors which include, for example, natural oils and fats from different organisms containing a considerable amount of the fatty acid which is to be converted by the fatty acid hydratase to the corresponding hydroxy fatty acid. Examples of natural oils and fats include soybean oil, corn oil, safflower oil, wheat germ oil, rice oil, sesame oil, rapeseed oil, olive oil, linseed oil, milk fat, suet, lard, egg yolk oil, fish oil, seaweed, algae, filamentous fungi, ferns and protozoa. Hydrolysates of natural oils and fats can be obtained by treating natural oils and fats with an enzyme such as a hydrolase, for example a lipase. The type of natural precursor to be used depends on the type of fatty acid which is to be reacted with the fatty acid hydratase. For example, linseed oil may be used as a natural precursor of linolenic acid, while sunflower oil may be used as a natural precursor of linoleic acid.

The fatty acid to be converted by the oleate hydratase of the present invention preferably has a length of between 12 and 22 carbon atoms, i.e. 12, 14, 16, 18, 20 or 22 carbon atoms, more preferably a length of between 12 and 18 carbon atoms, i.e. 12, 14, 16 or 18 carbon atoms. Most preferably the fatty acid to be converted by the enzyme of the present invention is oleic acid which has one double bond between carbon atoms 9 and 10 or its natural precursor, yielding upon hydration 10-hydroxyoctadecanoic acid.

Moreover, while polyunsaturated fatty acids (having two or more double bonds) can generally be used by the enzyme in the invention, it is preferred to use monounsaturated fatty acids (having only one double bond i.e. a (9Z) or (9E) double bond).

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences.

In other terms, using an alignment, for two or more sequences or subsequences the percentage of amino acid residues or nucleotides that are the same (e.g. 80% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Amino acid sequence analysis and alignment in connection with the present invention are preferably to be carried out using the NCBI BLAST algorithm (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) which is preferably employed in accordance with this invention. The skilled person is aware of additional suitable programs to align nucleic acid sequences.

As defined herein above, an amino acid sequence identity or a nucleotide sequence identity of at least 80% identity is envisaged by the invention. Furthermore are envisioned with increasing preference amino acid sequence identities or nucleotide sequence identities of at least 85%, at least 90%, at least 91%, at least 92%, at least 93%, at least 94%, at least 95%, at least 97.5%, at least 99%, at least 99.5%, at least 99.8% identity with the respective SEQ ID NO.

In order to arrive at the desired sequence identity, the nucleic acid or polypeptide may be modified using methods known in the art, such as, mutations or introduction of truncations, substitutions, deletions and/or additions. For example, a nucleic acid derived from Lactococcus spec. may be modified by altering the codons of the nucleic acid to reflect codon bias in an appropriate host cell and an oleate hydratase derived from Lactococcus may be modified by substituting amino acids. Also encompassed are allelic variants, which denotes any of two or more alternative forms of a gene occupying the same chromosomal locus. Allelic variation arises naturally through mutation, and may result in polymorphisms within populations. Gene mutations can be silent (no change in the encoded polypeptide) or may encode polypeptides having altered amino acid sequences. An allelic variant of a polypeptide is a polypeptide encoded by an allelic variant of a gene, wherein the allelic variant of the gene produces a change in the amino acid sequence of the polypeptide encoded therein.

Fragments according to the present invention are polypeptides having the activity of an oleate hydratase as defined herein above and comprising at least 400 amino acids or being encoded by at least 1200 nucleotides. In this regard, it is preferred with increasing preference that the fragments according to the present invention are polypeptides of at least 500, at least 550 and at least 585 amino acids (or encoded by at least 1500, at least 1650 and at least 1755 nucleotides), noting that SEQ ID NOs 1 and 7 comprise 587 and 590 amino acids, respectively (and SEQ ID NOs 2 and 8 comprise 1764 and 1773 nucleotides including the stop codons (TAA and TAG), respectively).

Fragments of the polypeptide of the invention, which substantially retain oleate hydratase activity, include N-terminal truncations, C-terminal truncations, amino acid substitutions, internal deletions and addition of amino acids (either internally or at either terminus of the protein). For example, conservative amino acid substitutions are known in the art and may be introduced into the oleate hydratase of the invention without substantially affecting oleate hydratase activity.

The amino acid sequence of SEQ ID NO: 3 is and the nucleotide sequence of SEQ ID NO: 4 encodes the oleate hydratase of Lactococcus lactis strain A106. In this respect it is of note that McCulloch et al. (2014), Genome Announcements, 2(6) sequenced and published the complete genome of Lactococcus lactis strain A106. While the amino acid sequence of SEQ ID NO: 3 and the nucleotide sequence of SEQ ID NO: 4 were published along with the genome as GenBank entry CP009472.1 it was erroneously published that SEQ ID NO: 3 is and SEQ ID NO: 4 encodes a linoleate isomerase. A linoleate isomerase (EC 5.2.1.5) is an enzyme that catalyzes the chemical reaction of 9-cis,12-cis-octadecadienoate into9-cis,11-trans-octadecadienoate. Hence, the enzyme transfers a cis double bond into a trans double bond rather than hydrolyzing a double bond into a single bond.

As shown herein below in Example 1, the fatty acid hydratase of Lactococcus lactis was cloned, sequenced and expressed. Moreover, it is demonstrated in Example 2 herein below that the fatty acid hydratase has in fact the activity of an oleate hydratase and not that of a linoleate isomerase. Hence, the present application surprisingly reveals for the first time that the amino acid sequence of SEQ ID NO: 3 is and the nucleotide sequence of SEQ ID NO: 4 as well as the related sequences as described herein above and retaining the oleate hydratase can be used for the production of a 10-HSA contrary to the discussed teaching in the prior art McCulloch et al. (2014), Genome Announcements, 2(6).

The amino acid sequence of SEQ ID NO: 1 is that of and the nucleotide sequence of SEQ ID NO: 2 encodes a chimeric oleate hydratase comprising the N-terminal part of the oleate hydratase of Lysinibacillus fusiformis and the C-terminal part of the oleate hydratase of Lactococcus lactis strain A106. In more detail, SEQ ID NO: 1 comprises 587 amino acids and the 193 N-terminal amino acids are from the N-terminus of the oleate hydratase of Lysinibacillus fusiformis (SEQ ID NO: 5) and the 394 C-terminal amino acids are from the C-terminus of the oleate hydratase of Lactococcus lactis (SEQ ID NO: 3). Similarly, SEQ ID NO: 2 comprises 1764 nucleotides and the 579 5′-nucleotides are from the 5′-end of the nucleotide sequence encoding the oleate hydratase of Lysinibacillus fusiformis (SEQ ID NO: 6) and the 1185 3′-nucleotides are from the 3′-end of the nucleotide sequence encoding the oleate hydratase of Lactococcus lactis (SEQ ID NO: 4). This chimeric oleate hydratase is also referred to herein as 1Lf-2L1.

The amino acid sequence of SEQ ID NO: 7 is that of and the nucleotide sequence of SEQ ID NO: 8 encodes a chimeric oleate hydratase comprising the N-terminal part of the oleate hydratase of Lactococcus lactis strain A106 and the C-terminal part of the oleate hydratase of Lysinibacillus fusiformis. In more detail, SEQ ID NO: 7 comprises 590 amino acids and the 193 N-terminal amino acids are from the N-terminus of the oleate hydratase of Lactococcus lactis (SEQ ID NO: 3) and the 397 C-terminal amino acids are from the C-terminus of the oleate hydratase of Lysinibacillus fusiformis (SEQ ID NO: 5). Similarly, SEQ ID NO: 8 comprises 1773 nucleotides and the 579 5′-nucleotides are from the 5′-end of the nucleotide sequence encoding the oleate hydratase of Lactococcus lactis (SEQ ID NO: 4) and the 1194 3′-nucleotides are from the 3′-end of the nucleotide sequence encoding the oleate hydratase of Lysinibacillus fusiformis (SEQ ID NO: 6). This chimeric oleate hydratase is also referred to herein as 1LI-2Lf.

The oleate hydratase of Lysinibacillus fusiformis is known from the prior art and has the amino acid sequence of SEQ ID NO: 5 and the nucleotide sequence of SEQ ID NO: 6.

Within the scope of the above described sequences sharing at least 80% sequence identity with SEQ ID NO: 1 or 7 and SEQ ID NO: 2 or 8 it is particularly preferred that they are chimeric oleate hydratases comprising the N-terminal part the oleate hydratase of Lysinibacillus fusiformis and the C-terminal part of the oleate hydratase of Lactococcus lactis strain A106 with the difference that the breakpoint is shifted. For example, in such a case the 587 amino acids may be composed of the 202 N-terminal amino acids from the N-terminus of the oleate hydratase of Lysinibacillus fusiformis and the 385 C-terminal amino acids from the C-terminus of the oleate hydratase of Lactococcus lactis strain A106. Similarly, in such a case the 587 amino acids may be composed of the 202 N-terminal amino acids from the N-terminus of the oleate hydratase of Lactococcus lactis strain A106 and the 385 C-terminal amino acids from the C-terminus of the oleate hydratase of Lysinibacillus fusiformis.

It is of further note that SEQ ID NO: 1 and SEQ ID NO: 3 share 89.6% identity and SEQ ID NOs 2 and 4 share 90.2% identity. For this reason SEQ ID NOs 3 and 4 are particularly preferred examples of sequences sharing at least 80% identity with SEQ ID NOs 1 and 2. Also sequences sharing with increasing preference at least 95%, at least 97%, at least 98%, at least 99%, and at least 99.5% sequence identity with SEQ ID NOs 3 and 4 are particularly preferred examples of sequences sharing at least 80% sequence identity with SEQ ID NOs 1 and 2. Also for this reason the amino acid sequence which is at least 80% identical to SEQ ID NO: 1 is preferably at least 89% identical to SEQ ID NO: 1 and the nucleotide sequence which is at least 80% identical to SEQ ID NO: 2 is preferably at least 90% identical to SEQ ID NO: 2.

In accordance with a preferred embodiment of the first aspect of the invention, the method further comprises the esterification of the 10-hydroxy fatty acid, thereby producing one or more esters of the 10-hydroxy fatty acid.

Esterification is the process of the conversion of an acid into an ester by combination with an alcohol and removal of a molecule of water. When the alcohol component is glycerol, the fatty acid esters produced can be monoglycerides, diglycerides, or triglycerides. Dietary fats are chemically triglycerides. In the present invention, 10-hydroxy fatty acid is converted into a 10-hydroxy fatty acid ester.

Methods for producing esters from fatty acids are known in the art and are, for example, described in Di Raddo (1993), J. Chem. Educ., 70(12):1034 or Marchetti and Errazu, Biomass and Bioenergy, 32(9):892-895.

Fatty acid esters are of commercial value. For example, biodiesels are typically fatty acid esters produced by the transesterification of vegetable fats and oils which results in the replacement of the glycerol component with a different alcohol. Further applications for fatty acids and esters are food stuff, cosmetics, soap and other personal care products, synthetic lubricants, paper, water treatment, as metal working fluids and in oil field applications.

In accordance with another preferred embodiment of the first aspect of the invention, the method further comprises the isolation of the 10-hydroxy fatty acid and/or the one or more esters thereof.

Methods of isolation of the 10-hydroxy fatty acid and/or the one or more esters thereof produced are well-known in the art and comprise without limitation method steps such as distillation, washing or extraction (e.g. by HPLC).

The step of the isolation of 10-hydroxy fatty acid and/or the one or more esters thereof is preferably a step of the purification of 10-hydroxy fatty acid and/or the one or more esters thereof. Purification in accordance with the invention specifies a process or a series of processes intended to further isolate the 10-hydroxy fatty acid and/or the one or more esters thereof of the invention from a complex mixture preferably to essentially 100% purity.

In accordance with another preferred embodiment of the first aspect of the invention, the step of contacting the sample comprising the (9Z) or (9E)-fatty acid with a polypeptide having the activity of an oleate hydratase (EC 4.2.1.53) is in the presence of flavin adenine dinucleotide (FAD) and/or reduced nicotinamide adenine dinucleotide (NADH).

Flavin adenine dinucleotide (FAD) is a redox-active coenzyme associated with various proteins, which is involved with several important enzymatic reactions in metabolism. FAD is not required for carrying out the method of the invention but its addition may accelerate the reaction speed.

FAD is preferably used in combination with NADH. When FAD and NADH coexist, the hydration activity of the fatty acid hydratase is expected to be further improved. The increased hydration activity may be due to the formation of reduced FAD (FADH2), the active cofactor, through the reduction of FAD by NADH, which allows an electronic complementation in the catalytic site of the enzyme.

If FAD and optionally also NADH are used FAD, is preferably used at about 0.1 mM and NADH at about 5 mM, wherein the term “about” is with increasing preference ±50%, ±25%, and ±10% (Kang et al., Appl Environ Microbiol. 2017 Apr 17;83(9)).

The present invention relates in a second aspect to the use of the polypeptide having the activity of an oleate hydratase (EC 4.2.1.53) as defined in connection with the first aspect of the invention for the production of a 10-hydroxy fatty acid.

The definitions and preferred embodiments as described herein above apply mutatis mutandis to the second aspect of the invention.

As discussed herein above, it is demonstrated in the appended examples for the first time that the polypeptide as defined in connection with the first aspect has the activity of an oleate hydratase and thus can be used for the production of a 10-hydroxy fatty acid.

In accordance with a preferred embodiment of the second aspect of the invention, the polypeptide having the activity of an oleate hydratase (EC 4.2.1.53) is used together with FAD and/or NADH.

As discussed herein above, the additional use of FAD and/or NADH, is not required but further accelerates the production of a 10-hydroxy fatty acid as catalyzed by the polypeptide encoded by the nucleic acid molecule in accordance with the invention.

In accordance with a preferred embodiment of the first and second aspect of the invention, (e) the nucleic acid molecule of claim 1c) encodes a polypeptide comprising or consisting of the amino acid sequence of SEQ ID NO: 3, and/or (d′) the nucleic acid molecule of claim 1d) comprises or consists of the nucleotide sequence of SEQ ID NO: 4.

As discussed, herein above the amino acid sequence of SEQ ID NO: 3 is and the nucleotide sequence of SEQ ID NO: 4 encodes the oleate hydratase of Lactococcus lactis strain A106.

The present invention relates in a third aspect to a nucleic acid molecule encoding a polypeptide having the activity of an oleate hydratase (EC 4.2.1.53), which nucleic acid molecule is (a) a nucleic acid molecule encoding a polypeptide comprising or consisting of the amino acid sequence of SEQ ID NO: 1 or 7; (b) a nucleic acid molecule comprising or consisting of the nucleotide sequence of SEQ ID NO: 2 or 8; (c) a nucleic acid molecule comprising or consisting of a nucleic acid molecule encoding a polypeptide having the activity of an oleate hydratase the amino acid sequence of which is at least 90%, preferably at least 91% identical to the amino acid sequence of SEQ ID NO: 1 or 7; (d) a nucleic acid molecule encoding a polypeptide having the activity of an oleate hydratase and comprising or consisting of a nucleotide sequence which is at least 91% identical to the nucleotide sequence of SEQ ID NO: 2 or 8; (e) a fragment of the nucleic acid molecule of one any of (a) to (d) comprising at least 1341 nucleotides and encoding a polypeptide having the activity of an oleate hydratase, or (f) the nucleic acid sequence of any of (a) to (d) wherein T is U.

The definitions and preferred embodiments as described herein above apply mutatis mutandis to the third aspect of the invention. For example, also in connection with the third aspect with increasing preference amino acid sequence identities or nucleotide sequence identities of at least 95%, at least 97.5%, at least 99%, at least 99.5%, and at least 99.8% identity are envisioned. Similarly, also in connection with the third aspect the fragments are with increasing preference polypeptides of at least 500, at least 550 and at least 585 amino acids (or encoded by at least at least 1500, at least 1650 and at least 1755 nucleotides).

As described herein above, the amino acid sequence of SEQ ID NO: 1 is and the nucleotide sequence of SEQ ID NO: 2 encodes a chimeric oleate hydratase comprising the N-terminal part of the oleate hydratase of Lysinibacillus fusiformis (as shown in SEQ ID NOs 5 and 6) and the C-terminal part of the oleate hydratase of Lactocoocus lactis strain A106 (as shown in SEQ ID NOs 3 and 4). As also discussed, SEQ ID NO: 1 and SEQ ID NO: 3 share 89.6% identity and SEQ ID NOs 2 and 4 share 90.2% identity.

As furthermore described herein above, the amino acid sequence of SEQ ID NO: 7 is and the nucleotide sequence of SEQ ID NO: 8 encodes a chimeric oleate hydratase comprising the N-terminal part of the oleate hydratase of Lactocoocus lactis strain A106 (as shown in SEQ ID NOs 3 and 4) and the C-terminal part of the oleate hydratase of Lysinibacillus fusiformis (as shown in SEQ ID NOs 5 and 6). SEQ ID NO: 7 and SEQ ID NO: 5 share 90.3% identity and SEQ ID NOs 8 and 6 share 90.5% identity.

To the best knowledge of the inventors neither an amino acid sequence which is at least 91% identical to SEQ ID NO: 1 or 7 nor a nucleotide sequence which is at least 91% identical to SEQ ID NO: 2 or 8 are known from the prior art.

Moreover, based on the most similar prior art sequences of SEQ ID NOs 3 and 4 which are erroneously reported in McCulloch et al. (2014), Genome Announcements, 2(6) and GenBank entry CP009472.1 to be or to encode a linoleate isomerase the skilled person would not have expected that SEQ ID NOs 1, 2 7 and 8 as well as sequences sharing at least 90% and at least 91% sequence identity therewith, respectively, display oleate hydratase activity.

Yet further, the results of example 3 herein below surprisingly reveal that the enzyme characteristics of both chimeric oleate hydratases 1Lf-2LI (SEQ ID NOs 1 and 2) or 1LI-2Lf (SEQ ID NOs 7 and 8) are significantly changed as compared to the wild-type hydratases used for the generation of the chimera. The examples herein below show that the activity of both chimeric enzymes at 30° C. was higher than the wildtype enzyme from Lactococcus lactis and lower than the wildtype enzyme from Lysinibacillus fusiformis (FIG. 3). Hence, the temperature profile of the constructed chimeric enzymes surprisingly changed compared to the wildtype enzymes. The 1Lf-2LI in addition offers a broader and higher enzyme activity at 20 to 25° C.

Moreover, the pH optimum is of this chimera slightly shifted to an acidic pH of 6.5. The described new features of both chimera significantly increase the biotechnological potential of the chimeras, thereby expanding their field of use. The new features provide operational advantages in technical processes. The thermostable chimeric oleate hydratases 1Lf-2L1 (SEQ ID NOs 1 and 2) or 1LI-2Lf (SEQ ID NOs 7 and 8) have longer operational stability at higher temperature and therefore advantageously offer robust catalyst alternatives capable of withstanding the comparatively stringent environments of industrial processing.The chimera of the invention may also be applied when the thermal stability of cold-adapted enzymes have to be improved. This is especially needed at critical temperatures at which non-cold-adapted enzymes begin to unfold.

In particular, the present invention provides chimeric oleate hydratases displaying an improved thermostability due to their change in the lower temperature range by the chimerization as illustrated in the examples. The use of enzymes that remain active at low temperatures has a great potential for industrial biocatalysis in terms of energy savings by lowering the required temperature of a reaction without sacrificing enzyme activity. The temperature adaptation of the catalytic properties has made cold-adapted enzymes promising biocatalysts for industrial applications, and they are now used in the synthesis of heat-labile fine chemicals, as additives in food processing at low temperatures, and in detergents for cold-water laundry.

The present invention relates in a fourth aspect to a polypeptide encoded by the nucleic acid molecule of the third aspect of the invention.

The definitions and preferred embodiments as described herein above apply mutatis mutandis to the fourth aspect of the invention. For example, the polypeptide of the fourth aspect shares with increasing preference at least 95%, at least 97.5%, at least 99%, at least 99.5 and at least 99.8% identity with SEQ ID NO: 2 or 8.

The polypeptide of the fourth aspect of the invention has the activity of an oleate hydratase (EC 4.2.1.53) and is thus an oleate hydratase.

The present invention relates in a fifth aspect to a fusion protein comprising the polypeptide of the third aspect of the invention.

The definitions and preferred embodiments as described herein above apply mutatis mutandis to the fifth aspect of the invention.

In addition to the amino acid sequence of the polypeptide of the present invention (which has the activity of an oleate hydratase), a fusion protein according to the present invention contains at least one additional, heterologous amino acid sequence. Often, but not necessarily, these additional sequences will be located at the N- or C-terminal end of the polypeptide. It may e.g. be convenient to initially express the polypeptide as a fusion protein from which the additional amino acid residues can be removed, e.g. by a proteinase (e.g. thrombin, factor VIII, factor Xa protease, or PreScission Protease) capable of specifically trimming the polypeptide of the present invention.

For example, the heterologous amino acid sequence may be a tag. Tags are attached to proteins for various purposes. Affinity tags are appended to proteins so that they can be purified from their crude biological source using an affinity technique. These include but are not limited to chitin binding protein (CBP), maltose binding protein (MBP), glutathione-S-transferase (GST), and poly(His) tag. The poly(His) tag is a widely used protein tag; it binds to metal matrices. Solubilization tags are used, especially for recombinant proteins expressed in chaperone-deficient species such as E. coli, to assist in the proper folding in proteins and keep them from precipitating. These include but are not limited to thioredoxin (TRX) and poly(NANP).

Some affinity tags have a dual role as a solubilization agent, such as MBP and GST. Chromatography tags are used to alter chromatographic properties of the protein to afford different resolution across a particular separation technique. Chromatography tags comprise but are not limited to of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include but are not limited to V5-tag, c-myc-tag, and HA-tag. These tags are particularly useful for western blotting and immunoprecipitation experiments, although they also find use in antibody purification. Fluorescence tags are used to give visual readout on a protein. For example, GFP and its variants are the most commonly used fluorescence tags. More advanced applications of GFP include using it as a folding reporter (fluorescent if folded, colorless if not). Moreover, tags find many other usages, such as specific enzymatic modification (such as biotin ligase tags) and chemical modification (FlAsH) tag. Examples of suitable tags to be used in accordance with the invention comprise but are not limited to lacZ, GST, maltose-binding protein, NusA, BCCP, c-myc, CaM, His, FLAG, GFP, YFP, cherry, thioredoxin, poly(NANP), V5, Snap, HA, chitin-binding protein, Softag 1, Softag 3, Strep, or S-protein.

The heterologous amino acid sequence may also be a protein which increases the solubility and/or facilitates the purification of the protein encoded by the nucleic acid molecule of the invention. Non-limiting examples include pET32, pET41, pET43.

Exemplary fusion proteins of the polypeptide of the invention will assist in expression and/or purification of the protein.

The present invention relates in a sixth aspect to a vector comprising the nucleic acid molecule of the third aspect of the invention.

The definitions and preferred embodiments as described herein above apply mutatis mutandis to the sixth aspect of the invention.

A vector according to this invention is generally and preferably capable of directing the replication, and/or the expression of the nucleic acid molecule of the invention and/or the expression of the polypeptide encoded thereby.

Preferably, the vector is a plasmid, cosmid, virus, bacteriophage or another vector used conventionally e.g. in genetic engineering.

The nucleic acid molecule of the present invention referred to above may also be inserted into vectors such that a translational fusion with another nucleic acid molecule is generated. To this aim, overlap extension PCR can be applied (e.g. Wurch, T., Lestienne, F., and Pauwels, P. J., A modified overlap extension PCR method to create chimeric genes in the absence of restriction enzymes, Biotechn. Techn. 12, 9, Sept. 1998, 653-657). The products arising therefrom are termed fusion proteins and have been described herein above. The vectors may also contain an additional expressible nucleic acid coding for one or more chaperones to facilitate correct protein folding. Suitable bacterial expression hosts comprise e. g. strains derived from BL21 (such as BL21(DE3), BL21(DE3)PlysS, BL21(DE3)RIL,

BL21(DE3)PRARE) or Rosetta®.

Particularly preferred plasmids which can be used to introduce the nucleic acid encoding the polypeptide of the invention having the activity of an oleate hydratase into the host cell are: pUC18/19 (Roche Biochemicals), pKK-177-3H (Roche Biochemicals), pBTac2 (Roche Biochemicals), pKK223-3 (Amersham Pharmacia Biotech), pKK-233-3 (Stratagene) and pET (Novagen). Further suitable plasmids are listed in PCT/EP03/07148. Very particular preference is given to an expression system which is based on plasmids belonging to the pET series.

For vector modification techniques, see Sambrook and Russel, 2001, Molecular cloning: a laboratory manual, 3^{rd} ed., Cold Spring Harbor Laboratory Press, New York. Generally, vectors can contain one or more origins of replication (on) and inheritance systems for cloning or expression, one or more markers for selection in the host, e.g., antibiotic resistance, and one or more expression cassettes. Suitable origins of replication include, for example, the Col E1, the SV40 viral and the M13 origins of replication.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, transcriptional termination sequences, promoters, enhancers, and/or insulators), internal ribosomal entry sites (IRES) and optionally poly-A signals ensuring termination of transcription and stabilization of the transcript. Additional regulatory elements may include transcriptional as well as translational enhancers, and/or naturally-associated or heterologous promoter regions. The regulatory elements may be native to the oleate hydratases of the invention or heterologous regulatory elements. Preferably, the nucleic acid molecule of the invention is operably linked to such expression control sequences allowing expression in prokaryotes or eukaryotic cells. The vector may further comprise nucleotide sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the nucleic acid molecule of the invention/used in accordance with the invention. Such leader sequences are well known in the art. Specifically designed vectors allow the shuttling of DNA between different hosts, such as bacteria-fungal cells or bacteria-animal cells. Expression vectors derived from viruses such as retroviruses, vaccinia virus, adeno-associated virus, herpes viruses, or bovine papilloma virus, may be used for delivery of the nucleic acids or vector into targeted cell population. Methods which are well known to those skilled in the art can be used to construct recombinant viral vectors; see, for example, the techniques described in Sambrook, 2001, Molecular cloning: a laboratory manual, 3^{rd} ed., Cold Spring Harbor Laboratory Press, New York.

The nucleic acid molecules of the invention/used in accordance with the invention as described herein above may be designed for direct introduction or for introduction via liposomes, phage vectors or viral vectors (e.g. adenoviral, retroviral) into the cell. Additionally, baculoviral systems or systems based on Vaccinia Virus or Semliki Forest Virus can be used as vector in eukaryotic expression system for the nucleic acid molecules of the invention/used in accordance with the invention.

Promoters which are particularly advantageous for implementing the invention and which are to be used, in particular, in E. coli are known to the skilled person (Sambrook, J.; Fritsch, E. F. and Maniatis, T. (1989), Molecular cloning: a laboratory manual, 2nd ed., Cold Spring Harbor Laboratory Press, New York). Further suitable promoters are those selected from T7, lac, tac, trp, ara or rhamnose-inducible promoters. Other promoters are mentioned in (Cantrell, S A (2003) Vectors for the expression of recombinant proteins in E. coli. Methods in Molecular biology 235: 257-275; Sawers, G; Jarsch, M (1996) Alternative principles for the production of recombinant proteins in Escherichia coli. Applied Microbiology and Biotechnology 46(1): 1-9). Examples for regulatory elements permitting expression in eukaryotic host cells are the AOX1 or GAL1 promoter in yeast or the CMV- (Cytomegalovirus), SV40-, RSV-promoter (Rous sarcoma virus), chicken beta-actin promoter, CAG-promoter (a combination of chicken beta-actin promoter and cytomegalovirus immediate-early enhancer), the gai 10 promoter, human elongation factor 1a-promoter, CMV enhancer, CaM-kinase promoter, the Autographa califomica multiple nuclear polyhedrosis virus (AcMNPV) polyhedral promoter or a globin intron in mammalian and other animal cells. The vectors may also comprise transcription termination signals, such as the SV40-poly-A site or the tk-poly-A site or the SV40, lacZ and AcMNPV polyhedral polyadenylation signals, downstream of the nucleic acid.

The co-transfection with a selectable marker such as kanamycin or ampicillin resistance genes for culturing in E. coli and other bacteria allows the identification and isolation of the transfected cells. Selectable markers for mammalian cell culture are the dhfr, gpt, neomycin, hygromycin resistance genes. Using such markers, the cells are grown in selective medium and the cells with the highest resistance are selected.

The present invention relates in a seventh aspect to a host cell carrying the vector of the sixth aspect of the invention.

The definitions and preferred embodiments as described herein above apply mutatis mutandis to the seventh aspect of the invention.

The host cell of the invention may “carry” the vector of the invention due to a transformation, transduction or transfection of the host cell with the vector. Accordingly, also describes herein is a host cell transformed, transduced or transfected with the vector of the invention.

Large amounts of the polypeptide of the fourth aspect of the invention may be produced by said transformed host, wherein the isolated nucleotide sequence encoding the polypeptide of the fourth aspect of the invention is inserted into an appropriate vector or expression vector before insertion into the host. The vector or expression vector is introduced into an appropriate host cell, which preferably can be grown in large quantities, and the polypeptide of the fourth aspect of the invention is purified from the host cells or the culture media.

The host cells may also be used to supply the polypeptide of the fourth aspect of the invention without requiring purification of the polypeptide of the fourth aspect of the invention (see Yuan, Y.; Wang, S.; Song, Z.; and Gao, R., Immobilization of an L-aminoacylase-producing strain of Aspergillus oryzae into gelatin pellets and its application in the resolution of D,L-methionine, Biotechnol Appl. Biochem. (2002). 35:107-113). For example, the polypeptide of the fourth aspect of the invention may be secreted by host cells, which are contacted with a hydrogen peroxide solution. Those skilled in the field of molecular biology will understand that any of a wide variety of expression systems may be used to provide the polypeptide of the fourth aspect of the invention. The precise host cell used is not critical to the invention, so long as the host cells produce the polypeptide of the fourth aspect of the invention when grown under suitable growth conditions.

Suitable prokaryotic host cells comprise e.g. bacteria of the species Escherichia, such as strains derived from E. coli BL21 (e.g. BL21(DE3), BL21(DE3)PlysS, BL21(DE3)RIL, BL21(DE3)PRARE, BL21 codon plus, BL21(DE3) codon plus), Rosetta®, XL1 Blue, NM522, JM101, JM109, JM105, RR1, DH5a, TOP 10, HB101 or MM294. Further suitable bacterial host cells are Streptomyces, Salmonella or Bacillus such as Bacillus subtilis. E. coli strains are preferred prokaryotic host cells in connection with the present invention.

Suitable eukaryotic host cells are e.g. yeasts such as Saccharomyces cerevisiae, Hansenula polymorpha or Pichia sp. such as Pichia pastoris, insect cells such as Drosophila S2 or Spodoptera Sf9 cells, plant cells, or fungi cells, preferably of the family Trichocomaceae, more preferably of the genus Aspergillus, Penicillium or Trichoderma reseei.

Mammalian host cells that could be used include human Hela, HEK293, H9 and Jurkat cells, mouse NIH3T3 and C127 cells, COS 1, COS 7 and CV1, quail QC1-3 cells, mouse L cells, Bowes melanoma cells and Chinese hamster ovary (CHO) cells.

The present invention relates in an eighth aspect to a method of producing a polypeptide having the activity of an oleate hydratase (EC 4.2.1.53) comprising (a) culturing the host cell of the seventh aspect of the invention, and (b) isolating the produced protein having the activity of an oleate hydratase.

The definitions and preferred embodiments as described herein above apply mutatis mutandis to the eighth aspect of the invention.

Suitable conditions for culturing a prokaryotic or eukaryotic host are well known to the person skilled in the art. Suitable conditions for culturing E. coli BL21 (DE3) are, for example provided in the examples of the specification. In general, suitable conditions for culturing bacteria are growing them under aeration in Luria Bertani (LB) medium. To increase the yield and the solubility of the expression product, the medium can be buffered or supplemented with suitable additives known to enhance or facilitate both. E. coli can be cultured from 4 to about 37 ° C. In general, Aspergillus sp. may be grown on Sabouraud dextrose agar, or potato dextrose agar at about to 10° C. to about 40° C., and preferably at about 25° C. Suitable conditions for yeast cultures are known, for example from Guthrie and Fink, “Guide to Yeast Genetics and Molecular Cell Biology” (2002); Academic Press Inc. The skilled person is also aware of all these conditions and may further adapt these conditions to the needs of a particular host species and the requirements of the polypeptide expressed. In case an inducible promoter controls the nucleic acid of the invention in the vector present in the host cell, expression of the polypeptide can be induced by addition of an appropriate inducing agent. Suitable expression protocols and strategies are known to the skilled person.

Depending on the cell type and its specific requirements, mammalian cell culture can e.g. be carried out in RPMI or DMEM medium containing 10% (v/v) FCS, 2 mM L-glutamine and 100 U/ml penicillin/streptomycin. The cells can be kept at 37° C. in a 5% CO₂, water saturated atmosphere. Suitable expression protocols for eukaryotic cells are well known to the skilled person and can be retrieved e.g. from in Sambrook, 2001

Suitable media for insect cell culture is e.g. TNM+10% FCS or SF900 medium. Insect cells are usually grown at 27° C. as adhesion or suspension culture.

Methods of isolation of the polypeptide produced are well-known in the art and comprise without limitation method steps such as ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, Sambrook, 2001.

The step of protein isolation is preferably a step of protein purification. Protein purification in accordance with the invention specifies a process or a series of processes intended to further isolate the polypeptide of the invention from a complex mixture preferably to homogeneity. Purification steps, for example, exploit differences in protein size, physico-chemical properties and binding affinity. For example, proteins may be purified according to their isoelectric points by running them through a pH graded gel or an ion exchange column. Further, proteins may be separated according to their size or molecular weight via size exclusion chromatography or by SDS-PAGE (sodium dodecyl sulfate-polyacrylamide gel electrophoresis) analysis. In the art, proteins are often purified by using 2D-PAGE and are then further analysed by peptide mass fingerprinting to establish the protein identity. The detection limits for protein are very low and nanogram amounts of protein are sufficient for their analysis. Proteins may also be separated by polarity/hydrophobicity via high performance liquid chromatography or reversed-phase chromatography. Thus, methods for protein purification are well known to the skilled person and are exemplified in the examples of the invention.

The present invention relates in a ninth aspect to a composition comprising the nucleic acid molecule of the third aspect, the polypeptide of the fourth aspect, the fusion protein of the fifth aspect, the vector of the sixth aspect, the host cell of the seventh aspect of the invention, or combinations thereof.

A composition refers to any mixture of ingredients, wherein at least one ingredient is in accordance with the invention the nucleic acid molecule of the third aspect, the polypeptide of the fourth aspect, the fusion protein of the fifth aspect, the vector of the sixth aspect, the host cell of the seventh aspect of the invention, or combinations thereof.

The other compound may be, for example, any diluent or carrier of the nucleic acid molecule of the third aspect, the polypeptide of the fourth aspect, the fusion protein of the fifth aspect, the vector of the sixth aspect, the host cell of the seventh aspect of the invention, or combinations thereof.

The composition may be a large-scale composition. In accordance with the invention a “large-scale composition” refers to a composition involved in the production of an economic good within an economy.

Moreover, the composition is preferably selected from a lubricant, a surface coating, a plastic, a resin, a biodiesel, a detergent, a paint, an organogel, and a precursor composition of lactones.

In accordance with a preferred embodiment of the ninth aspect of the invention, the composition is a food composition, a cosmetic composition, a pharmaceutical composition, or a diagnostic composition.

The definitions and preferred embodiments as described herein above apply mutatis mutandis to the ninth aspect of the invention.

As discussed above, fatty acids and esters are used in food stuff, cosmetics, soap and other personal care products, synthetic lubricants, paper, water treatment, as metal working fluids and in oil field applications. Since the nucleic acid molecule of the third aspect, the polypeptide of the fourth aspect, the fusion protein of the fifth aspect, the vector of the sixth aspect, the host cell of the seventh aspect of the invention, or combinations thereof can be used to produce 10-HSA and esters thereof they may be used in compositions being required to produce such economic goods.

In accordance with the present invention, the term “food composition” relates to any composition which is edible or drinkable and provide values for energy and nutrients when consumed by a subject, in particular a human. Non-limiting examples of food compositions are beverages, natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, meat and foods boiled in sweetened sauce, agricultural vegetable food products, seafood, ham, sausage, fish ham, fish sausage, fish paste, deep fried fish products, dried seafood products, frozen food products, preserved seaweed, and preserved meat.

Certain fatty acids (e.g. linoleic acid) cannot be made by the body and therefore must be taken in the diet. Although data on the required intake of essential fatty acids are relatively few, the adequate intakes of linoleic acid and a-linolenic acid should be 2% and 1% of total energy, respectively. Present evidence suggests that 0.2-0.3% of the energy should be derived from preformed very long-chain omega-3 PUFAs (EPA and DHA) to avoid signs or symptoms of deficiency. By the polypeptide of the invention it is now possible to modify the fatty acid composition of, for example, plant oilseeds, which opens up the possibility of improving the nutritional quality and to prevent or treat symptoms of deficiency.

In accordance with the present invention, the term “pharmaceutical composition” relates to a composition for administration to a patient, preferably a human patient. The pharmaceutical composition of the invention comprises the compounds recited above, alone or in combination. The composition may be in solid or liquid form and may be, inter alia, in the form of (a) powder(s), (a) solution(s) or (an) aerosol(s), cream(s), ointment(s) or gel(s). The pharmaceutical composition of the present invention may, optionally and additionally, comprise a pharmaceutically acceptable carrier. By “pharmaceutically acceptable carrier” is meant a non-toxic solid, semisolid or liquid filler, diluent, encapsulating material or formulation auxiliary of any type. Examples of suitable pharmaceutical carriers are well known in the art. Compositions comprising such carriers can be formulated by well known conventional methods. The pharmaceutical composition can be administered topically. The dosage regimen corresponding to a suitable dose for administration will be determined by the attending physician and clinical factors which may, inter alia, depend on the size of the area to be treated, the stage or severity of its condition. The concentration of the compound(s) as recited above in a composition for topical application can be in the range of 0.001 to 1% (w/w), preferably 0.01-0.1% (w/w). Topical application is preferably repeated in one or more than one daily applications.

The pharmaceutical composition of the invention can be applied in combination with (solid) carriers or matrices such as dressing(s), band aid(s) or tape(s). The compound(s) can be covalently or non-covalently bound to said carrier or matrix. For example, the compound(s) may be incorporated into a dressing to be applied over the area to be treated. Examples of such dressings include staged or layered dressings incorporating slow-release hydrocolloid particles. The concentration of a solution of the pharmaceutical composition to be immobilised in a matrix of a dressing is generally in the range of 0.001 to 1% (w/v) preferably 0.01-0.1% (w/v). Furthermore, the compound(s) as recited above can be incorporated into a suitable material capable of delivering the enzyme to the area to be treated in a slow release or controlled release manner.

A gel formulation of the pharmaceutical composition of the present invention further comprises at least one gel forming agent commonly used in pharmaceutical gel formulations. Examples of gel forming agents are cellulose derivatives such as methyl cellulose, hydroxyethyl cellulose, and carboxymethyl cellulose; vinyl polymers such as polyvinyl alcohols, polyvinyl pyrrolidones; and carboxypoly-methylene derivatives such as carbopol. Further gelling agents that can be used for the present invention are pectins, gums, alginates, agar and gelatine. Furthermore, the gel or emugel formulation may contain auxiliary agents commonly used in this kind of formulations such as preservatives, antioxidants, stabilizers, colorants and perfumes.

A diagnostic composition according to the invention is for use in the detection of a disease in a subject or the risk of developing a disease in a subject. The disease or risk for developing the disease is preferably identified in a sample obtained from the subject to be diagnosed. The sample is not particularly limited and may be any tissue or body fluid sample. The body fluid sample is preferably a blood sample, such as whole blood, serum or plasma. The disease to be diagnosed is preferably a metabolic disorder or a microbiological, such as a bacterial infection. Accordingly, the composition of the invention as described herein above may also be used non-therapeutically for the detection of a microbiological contamination in consumable goods, such as cosmetics, food or beverages.

A cosmetic composition according to the invention is for use in non-therapeutic applications. Cosmetic compositions may also be defined by their intended use, as compositions intended to be rubbed, poured, sprinkled, or sprayed on, or otherwise applied to the human body for cleansing, beautifying, promoting attractiveness, or altering the appearance.

The particular formulation of the cosmetic composition according to the invention is not limited. Envisaged formulations include rinse solutions, emulsions, creams, milks, gels such as hydrogels, ointments, suspensions, dispersions, powders, solid sticks, foams, sprays and shampoos. For this purpose, the cosmetic composition according to the invention may further comprise cosmetically acceptable diluents and/or carriers. Choosing appropriate carriers and diluents in dependency of the desired formulation is within the skills of the skilled person. Suitable cosmetically acceptable diluents and carriers are well known in the art and include agents referred to in Bushell et al. (WO 2006/053613). Preferred formulations for said cosmetic composition are rinse solutions and creams.

The application of the composition of the invention in cosmetics is, for example, aiming at treating the skin and skin appendages (e.g. hair and nails) enzymatically for converting hydrogen peroxide into water and oxygen. A suitable concentration of the compound(s) of the invention for cosmetic use is believed to be in the range of 0.0001 to 1% (w/v), preferably 0.0001 to 0.1% (w/v), even more preferably 0.001 to 0.1% (w/v).

Preferred amounts of the cosmetic compositions according to the invention to be applied in a single application are between 0.1 and 10 g, more preferred between 0.1 and 1 g, most preferred 0.5 g. The amount to be applied also depends on the size of the area to be treated and has to be adapted thereto.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

### THE FIGURES SHOW

FIG. 1: Hydratase reaction of oleic acid to 10-hydroxystearic acid catalyzed by fatty acid hydratase.

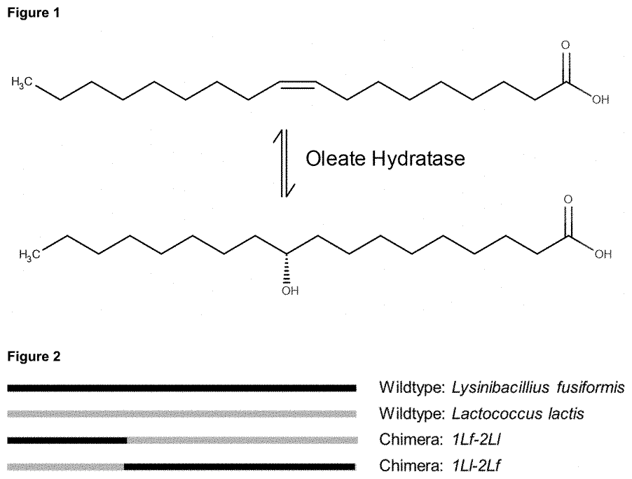

FIG. 2: Construction of chimeric fatty acid hydratases from Lysinibacillus fusiformis and Lactococcus lactis

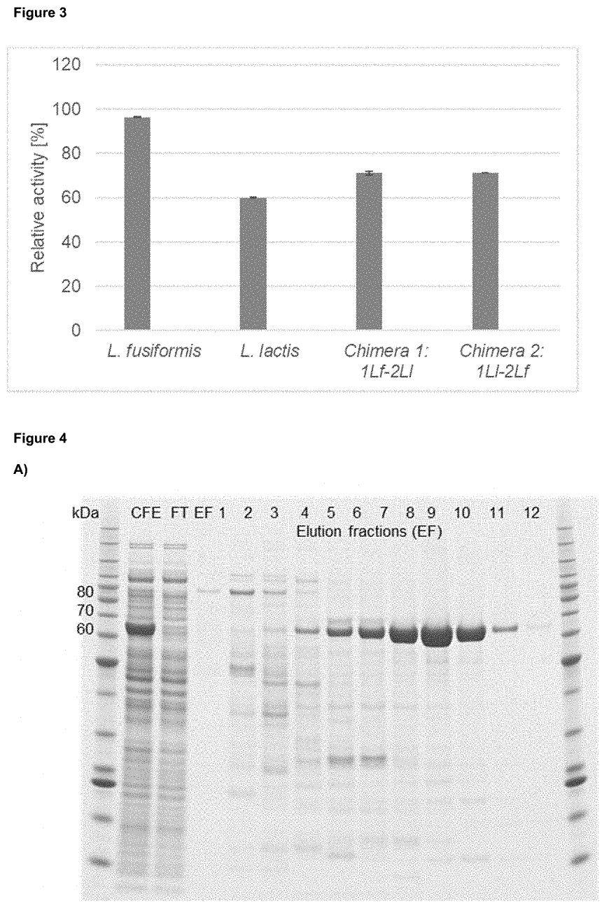

FIG. 3: Conversion of oleic acid to 10-hydroxystearic acid by fatty acid hydratases from Lysinibacillus fusiformis and Lactococcus lactis

FIG. 4: (A) SDS-PAGE (Coomassie stain) of oleate hydratase from Lactococcus lactis purified by Ni-NTA affinity chromatography showing the apparent molecular weight of approx. 68 kDa. 10 μL of cell-free extract (CFE), 10 μL of flow through (FT), 10 μL of concentrated elution fraction (EF) 1-12 were loaded onto an SDS-PA gel for providing correct size of approximately 68 kDa and high purity of the eluted protein. (B) SDS-PAGE (Coomassie stain) of chimeric oleate hydratase from Lysinibacillus fusiformis and Lactococcus lactis and purified by Ni-NTA affinity chromatography showing the apparent molecular weight of approx. 68 kDa. 10 μL of cell-free extract (CFE), 10 μL of flow through (FT), 10 μL of concentrated elution fraction (EF) 1-11 were loaded onto an SDS-PA gel for providing correct size of approximately 68 kDa and high purity of the eluted protein. (B) SDS-PAGE (Coomassie stain) of chimeric oleate hydratase from Lactococcus lactis and Lysinibacillus fusiformis showing the apparent molecular weight of approx. 67 kDa. 10 μL of cell-free extract (CFE), 10 μL of insoluble fraction (IF).

FIG. 5: Conversion of oleic acid to 10-hydroxystearic acid by fatty acid hydratase from Lactococcus lactis regarding pH profile (A) and temperature (B). FIG. 6: Conversion of oleic acid to 10-hydroxystearic acid by chimeric fatty acid hydratases from Lysinibacillus fusiformis and Lactococcus lactis regarding pH profile (A) and temperature (B).

The following Examples illustrate the invention.

### Example 1

### Cloning, Expression and Purification of the Fatty Acid Hydratases of Lactococcus lactis and Lysinibacillus fusiformis

Genomic DNA of Lactococcus lactis and Lysinibacillus fusiformis were isolated and used for a PCR screening with degenerated primers. The genes encoding fatty acid hydratases (EC 4.2.1.53) of Lactococcus lactis and Lysinibacillus fusiformis, respectively, were cloned in a pET26 expression vector, with the addition of a methionine initiation codon and a 6-histidine tag added at the C-terminal end. Overlap extension PCR technique was used for the creation of the chimeric enzyme. The first step was a conventional PCR reaction, in which oligonucleotide primers were partially complementary at their 5′ ends to the respective adjacent fragment which was subsequently fused to create the chimera. The reverse primer of fragment 1 Lf (N-terminal sequence from Lysinibacillus fusiformis) was complementary at its 5′ end to the 5′ end of the forward primer of fragment 2LI (C-terminal sequence from Lactococcus lactis).

The second PCR step consisted in the fusion of the PCR fragments generated in the first step using the complementary extremities of the primers. In the third step the fusion product was amplified by PCR.

In yet another example, the chimeric enzyme comprises an N-terminal fragment of Lactoccous lactis (fragment 1L1) and a C-terminal fragment of Lysinibacillus fusiformis (fragment 2Lf) The fusion site of the fragments 1LI and 2Lf is at the same amino acid position as for the chimeric enzyme described above (fragment 1Lf-2L1).

Competent E. coli BL21 (DE3) cells (Novagen) were transformed with these vectors by heat shock. The recombinant E. coli cells for protein expression were cultivated in a 2,000-ml flask containing 200 ml of Luria—Bertani (LB) medium and 25 μg ml⁻¹ of kanamycin at 37° C. with shaking at 200 rpm. When the optical density of the bacterial culture at 600 nm reached 0.6, isopropyl-11-D-thiogalactopyranoside was added to a final concentration of 0.1 mM to induce enzyme expression, and the culture was incubated with shaking at 200 rpm at 25° C. for 16 h. The cells were collected by centrifugation at 4° C., 10.000 rpm for 20 min and the pellets were frozen at −80° C.

Cell-free extracts of the wild type enzymes and the chimera from Lysinibacillus fusiformis and Lactococcus lactis were assayed in 100 mM citrate/phosphate buffer (pH7.0) containing 10 mM MgSO₄ at 30° C. FAD (0.1 mM) and NADH (5 mM) were applied for the FAD-reducing conditions. Reactions were started by the addition of 150 mM oleic acid for 60 min. The conversion of oleic acid to 10-hydroxystearic acid was confirmed by HPLC analysis for both wild type and both chimeric enzymes (oriented either fragment 1Lf-2LI or fragment 1LI-2Lf).

### Example 2

### Cell Lysis And Protein Purification of Fatty Acid Hydratase from Lactococcus lactis and Chimeric Oleate Hydratase from Lysinibacillus fusiformis and Lactococcus lactis

For protein purification cell lysates were obtained by resuspension of the cell pellet in buffer A (50 mM piperazine-N,N′-bis-(2-ethanesulfonic acid) (PIPES) buffer (pH 6.5) containing 1 mM CaCl₂, 300 mM NaCl and 15 mM imidazole). The resuspended cells were disrupted using Branson Ultrasonics^{TM} Sonifier S-250 (Branson Ultrasonics™ Cooperation, Danbury, Con., USA) at duty cycle 50%, output control 5.5 for 1 min, six times on ice.

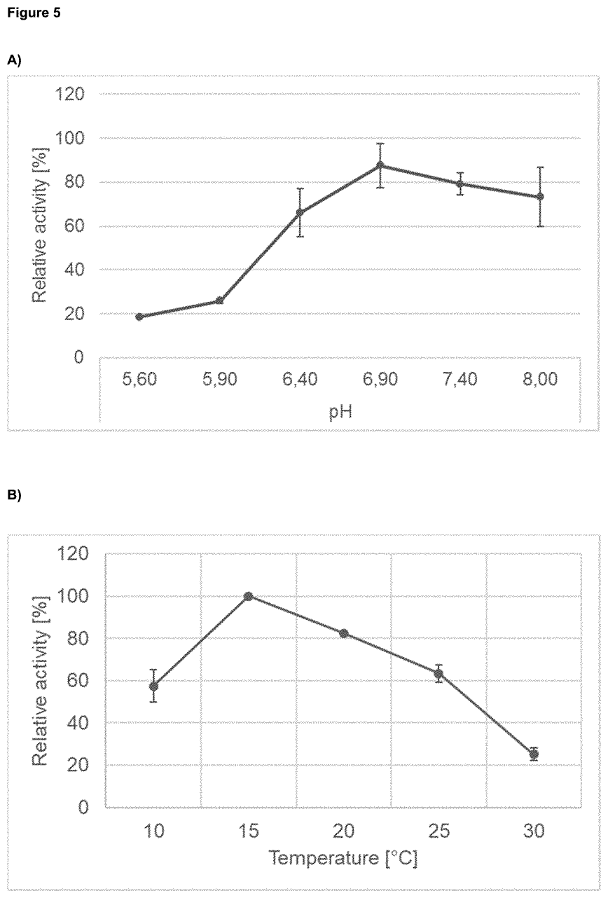

The cell debris was removed by centrifugation at 3,894×g for 30 min at 4° C., and the supernatant was filtered through a 0.45-μm filter. The filtrate was applied to a His-Trap HP chromatography column (Amersham Biosciences, Uppsala, Sweden) equilibrated with 50 mM piperazine-N,N′-bis-(2-ethanesulfonic acid) (PIPES) buffer (pH 6.5) containing 1 mM CaCl₂, 300 mM NaCl, 15 mM imidazole. The column was equilibrated with 10 column volumes of buffer A, clear supernatant was loaded at 1 ml/min, column washed with 10 column volumes buffer A and protein eluted in buffer B (buffer A with 0.3 M imidazol). Fractions of 1 ml were collected. After elution 10 μl aliquots of peak fractions were tested by SDS PAGE. The active fractions were collected and immediately buffer exchanged via disposable PD-10 desalting columns (GE Healthcare, UK) according to the recommended protocol. The resultant solution was used as the purified enzyme. Proteins were quantified by the BCA method. SDS-PAGE analyses were conducted in parallel: a main band is clearly visible around 68 kDa, and the purity of the protein is estimated to over 80% (FIG. 4).

### Example 3

### Effects of pH and Temperature on Enzyme Activity of Fatty Acid Hydratase from Lactococcus lactis and Chimeric Oleate Hydratase from Lysinibacillus fusiformis and Lactococcus lactis

### (A) Effects of pH

The reactions were performed in 100 mM citrate/phosphate buffer (pH levels ranging from 5.6-8.0) containing MgSO4; 8.9 mg/ml total protein of Lactococcus lactis or 9.3 mg/mL of total protein of chimeric oleate hydratase from Lysinibacillus fusiformis and Lactococcus lactis. FAD (0.1 mM) and NADH (5 mM) were applied for the FAD-reducing conditions. Reactions were started by the addition of 150 mM oleic acid at 30° C. for 40 min. Following the reactions, the solutions of fatty acids and hydroxy fatty acids were recovered by three consecutive extractions with 1.6 volume of dichloromethane. After solvent evaporation, the resultant sample was diluted in ethanol and analyzed by HPLC-MS. Analyses were performed on an Agilent 1100 HPLC instrument equipped with evaporative light scattering detector (ELSD) and a Luna® C18 (2) HPLC column (RP-18e 5 μm, 250×4.6 mm) maintained at 50° C. The elution system consisted of ddH₂O with 0.1% formic acid (A) and methanol with 0.1% formic acid (B). The gradient was set as follows: 0 min (80% B); 15 min (100% B); 23.2 min (80% B) at a flow rate of 0.7 mL min⁻¹.

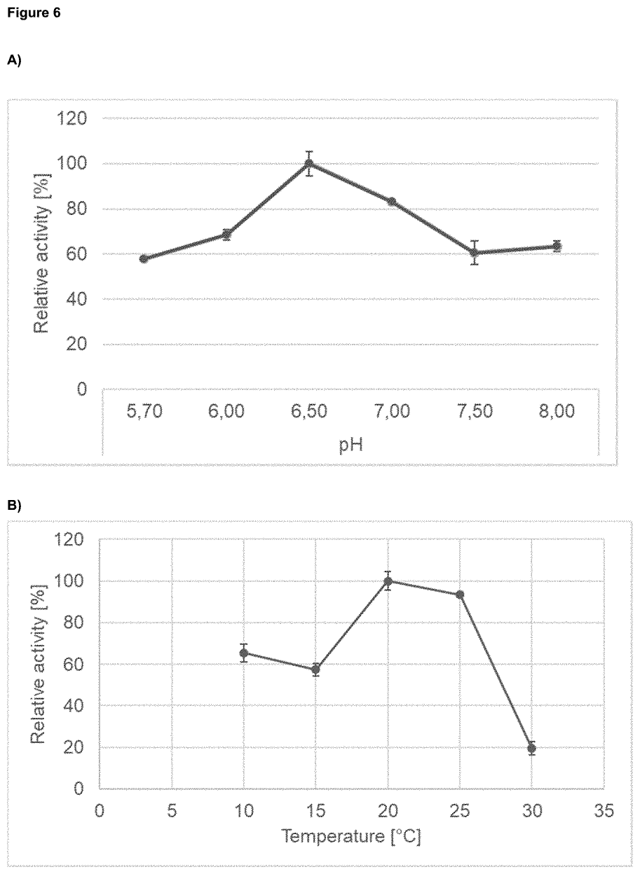

Data represent the mean±standard deviation of three independent experiments (FIG. 5A and FIG. 6A).

The pH optimum of the oleate hydratase from Lactococcus lactis was in the range of 6.9 to 8.0 (FIG. 5A) whereas the pH profile of the chimeric oleate hydratase from Lysinibacillus fusiformis and Lactococcus lactis was shifted to a slightly lower pH optimum at 6.5 with decreasing activity at higher pH values (FIG. 6A).

### (B) Effects of Temperature

The reactions were performed in 100 mM citrate/phosphate buffer (pH7.0) containing MgSO₄;

3.1 mg/ml total protein of Lactococcus lactis (a) or 5.6 mg/mL of total protein of chimeric oleate hydratase from Lysinibacillus fusiformis and Lactococcus lactis (b). FAD (0.1 mM) and NADH (5 mM) were applied for the FAD-reducing conditions. Reactions were started by the addition of 150 mM oleic acid for 20 min. At the relative activity of 100%, the specific enzyme activity of the oleate hydratase from Lactococcus lactis was 0.8 μmol min⁻¹ mg⁻¹ total protein and the specific enzyme activity of the chimeric oleate hydratase from Lysinibacillus fusiformis and Lactococcus lactis was 1.0 μmol min⁻¹ mg⁻¹ total protein.

The oleate hydratase from Lactococcus lactis has its highest enzyme activity at 15° C. Maximal enzyme activity of the oleate hydratase from Lysinibacillus fusiformis was observed at 35° C. (Kim, Bi-Na et al. (2012) Appl. Microbiol. Biotechnol. 95, 929-937).

The chimeric enzyme 1 Lf-2L1 showed a surprisingly broader and higher temperature optimum of enzyme activity at 20° C. -25° C. (FIG. 6 B) than the wildtype enzyme from Lactococcus lactis (FIG. 5B).

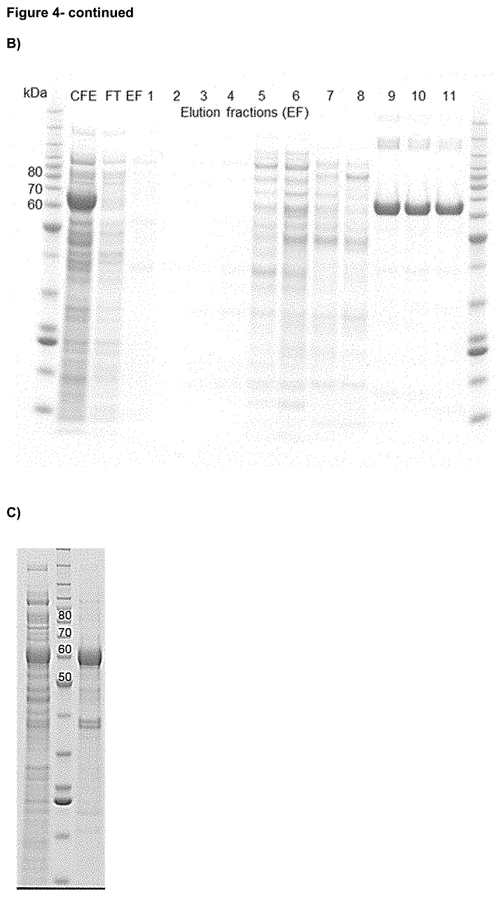

The activity of both chimeric enzymes at 30° C. was higher than the wildtype enzyme from Lactococcus lactis and lower than the wildtype enzyme from Lysinibacillus fusiformis (FIG. 3). The temperature profile of the constructed chimeric enzymes surprisingly changed compared to the wildtype. The recombination of oleate hydratase fragments from different microbial flora unexpectedly enables the development of enzymes for different application fields. The use of enzymes that remain active at low temperatures has a great potential for industrial biocatalysis in terms of energy savings by lowering the required temperature of a reaction without sacrificing enzyme activity. The temperature adaptation of the catalytic properties has made cold-adapted enzymes promising biocatalysts for industrial applications, and they are now used in the synthesis of heat-labile fine chemicals, as additives in food processing at low temperatures, and in detergents for cold-water laundry. Chimerization may also be applied when the thermal stability of cold-adapted enzymes, especially at critical temperatures at which the enzymes begin to unfold, have to be improved.

The results of examples 1 and 3 show that the enzyme characteristics were changed significantly by creating a chimeric oleate hydratase using fragments of two wildtype enzymes from different microbial species. The new features offered by chimerization significantly increase the biotechnological potential of this biocatalyst, expanding its field of application and provide energetic advantages in technical processes which can be performed at ambient temperatures using the enzyme of the invention.

1. A nucleic acid molecule encoding a polypeptide having the activity of an oleate hydratase (EC 4.2.1.53), which nucleic acid molecule is
(a) a nucleic acid molecule encoding a polypeptide comprising or consisting of the amino acid sequence of SEQ ID NO: 1 or 7;
(b) a nucleic acid molecule comprising or consisting of the nucleotide sequence of SEQ ID NO: 2 or 8;
(c) a nucleic acid molecule comprising or consisting of a nucleic acid molecule encoding a polypeptide having the activity of an oleate hydratase the amino acid sequence of which is at least 91% identical to the amino acid sequence of SEQ ID NO: 1 or 7;
(d) a nucleic acid molecule encoding a polypeptide having the activity of an oleate hydratase and comprising or consisting of a nucleotide sequence which is at least 91% identical to the nucleotide sequence of SEQ ID NO: 2 or 8;
(e) a fragment of the nucleic acid molecule of one any of (a) to (d) comprising at least 1200 nucleotides and encoding a polypeptide having the activity of an oleate hydratase; or
(f) the nucleic acid sequence of any of (a) to (d) wherein T is U.

2. A polypeptide encoded by the nucleic acid molecule of claim 1.

3. A fusion protein comprising the polypeptide of claim 2.

4. A vector comprising the nucleic acid molecule of claim 1.

5. A host cell carrying the vector of claim 4.

6. A method of producing a polypeptide having the activity of an oleate hydratase (EC 4.2.1.53) comprising
(a) culturing the host cell of claim 5, and
(b) isolating the produced protein having the activity of an oleate hydratase.

7. A composition comprising the nucleic acid molecule of claim 1.

8. The composition of claim 7, which is a large-scale composition, a food composition, a cosmetic composition, a pharmaceutical composition, or a diagnostic composition.

9. A method of producing a 10-hydroxy fatty acid, wherein the method comprises contacting a sample comprising a (9Z) or (9E)-fatty acid with a polypeptide having the activity of an oleate hydratase (EC 4.2.1.53) encoded by a nucleic acid molecule, wherein the nucleic acid molecule is
(a) a nucleic acid molecule encoding a polypeptide comprising or consisting of the amino acid sequence of SEQ ID NO: 1 or 7;
(b) a nucleic acid molecule comprising or consisting of the nucleotide sequence of SEQ ID NO: 2 or 8;
(c) a nucleic acid molecule comprising or consisting of a nucleic acid molecule encoding a polypeptide having the activity of an oleate hydratase the amino acid sequence of which is at least 91% identical to the amino acid sequence of SEQ ID NO: 1 or 7;
(d) a nucleic acid molecule encoding a polypeptide having the activity of an oleate hydratase and comprising or consisting of a nucleotide sequence which is at least 91° A identical to the nucleotide sequence of SEQ ID NO: 2 or 8;
(e) a fragment of the nucleic acid molecule of any of (a) to (d) comprising at least 1200 nucleotides and encoding a polypeptide having the activity of an oleate hydratase; or
(f) the nucleic acid sequence of any of (a) to (d) wherein T is U.

10. The method of claim 9, further comprising the esterification of the 10-hydroxy fatty acid, thereby producing one or more esters of the 10-hydroxy fatty acid.

11. The method of claim 9 or 10, further comprising the isolation of the 10-hydroxy fatty acid.

12. The method of claim 9, wherein the step of contacting the sample comprising the (9Z) or (9E)-fatty acid with a polypeptide having the activity of an oleate hydratase (EC 4.2.1.53) is in the presence of flavin adenine dinucleotide (FAD) and/or reduced nicotinamide adenine dinucleotide (NADH).

13. Use of the polypeptide having the activity of an oleate hydratase (EC 4.2.1.53) as defined in claim 9 for the production of a 10-hydroxy fatty acid.

14. The use of claim 13, wherein the polypeptide having the activity of an oleate hydratase (EC 4.2.1.53) is used together with FAD and/or NADH.

15. The method of claim 9, wherein
(c′) the nucleic acid molecule of (c) encodes a polypeptide comprising or consisting of the amino acid sequence of SEQ ID NO: 3, and/or
(d′) the nucleic acid molecule of (d) comprises or consists of the nucleotide sequence of SEQ ID NO: 4.

16. A composition comprising the polypeptide of claim 2.

17. A composition comprising the fusion protein of claim 3.

18. A composition comprising the vector of claim 4.

19. A composition comprising the host cell of claim 5.

20. The method of claim 10, further comprising the isolation of the one or more esters of the 10-hydroxy fatty acid.
